(19) United States
(12) Patent Application Publication
(10) Pub. No.: US 2013/0345592 A1
(43) Pub. Date: Dec. 26, 2013

(54) METHOD AND UNIT FOR DETERMINING BODY COMPOSITION PARAMETERS WITH THE AID OF BIOIMPEDANCE MEASUREMENT

(71) Applicants: Rudiger Leuner, Hamburg (DE); Anja Bosy-Westphal, Stuttgart (DE)

(72) Inventors: Rudiger Leuner, Hamburg (DE); Anja Bosy-Westphal, Stuttgart (DE)

(21) Appl. No.: 13/627,212

(22) Filed: Sep. 26, 2012

(30) Foreign Application Priority Data
Jun. 26, 2012 (EP) 12173620.1

(51) Int. Cl. A61B 5/053

(52) U.S. Cl. CPC A61B 5/0537
USPC 600/547

(57) ABSTRACT

The disclosure relates to a method for determining a body composition parameter by applying a bioimpedance measurement on a subject, the overall body size and an impedance value of the body of the subject entering into the calculation of the body composition parameter. It is provided that when calculating the body composition parameter a term is additionally entered which constitutes a product of a previously determined coefficient and of a ratio of measured impedance values of different body segments of the subject. During the calculation of the ratio of impedance values of different body segments the ratio of the real parts of the impedances preferably takes the form of

$${x}_{R Trunk / Extremities} = \frac{{R}_{Trunk}}{( {R}_{Right arm} + {R}_{Left arm} + {R}_{Right leg} + {R}_{Left leg} ) / 4} .$$

### CROSS REFERENCE TO RELATED APPLICATIONS

This application claims priority from European Patent Application No. 12173620.1, filed Jun. 26, 2012, the entire disclosure of which is hereby incorporated by reference herein.

### BACKGROUND OF THE INVENTION

1. Field of the Invention

The present invention relates to a method and a unit for determining a body composition parameter by applying a bioimpedance measurement on a subject, the overall body size and an impedance value of the body of the subject entering into the calculation of the body composition parameter.

2. Discussion of the Prior Art

The conductivity of the human body is strongly influenced by the water component. Since the fat-free components of the body such as muscles and the body fluids include a major part of the endogenous water, while fat tissue has an only relatively small water component, the determination of the conductivity of the body or of a body segment (or, conversely, of the resistance or of the impedance of the body or of the body segment) can allow a conclusion to be drawn on the relative proportion of fat, at least when further data, such as the size and weight of the person, are taken into account.

A method and a device for bioimpedance analysis are described, for example, in WO 97/01303. The described device has eight electrodes, specifically four foot electrodes, respectively two for making contact with a foot, and four hand electrodes, respectively two for making contact with a hand of the person. An alternating current is then injected via respectively two electrodes located on different limbs, and the voltage is measured at two electrodes, likewise present on different limbs. Successive different body segments can be examined by transition to other pairs of current-injecting electrodes and voltage-detecting electrodes. Furthermore, an entire body side can be measured in the case of the feeding of current into a hand and a foot and measuring voltage on the same hand and on the same foot.

Body composition parameters in the sense of the present description are, for example, the fat-free mass (FFM), the fat mass (FM), the total body water mass (TBW), extracellular water (ECW) or lean soft tissue mass (LST).

Methods of the type mentioned at the beginning are, for example, described in the article entitled “Bioelectrical impedance analysis—part I: review of principles and methods” by Ursula G. Kyle et al., Espen Guidelines. In such methods for determining body composition parameters while taking account of impedance measurements, the values of a body composition parameter are calculated with the aid of an equation of the form:

Value=a₁·x₁+a₂·x₂+ . . . ,

wherein some xᵢ are factors which include the measured values for the impedances of the body, and the coefficients aᵢ are predetermined coefficients. These coefficients are determined in studies in which the values of the body composition parameter are determined using independent methods for a representative group of subjects, and impedance measurements are carried out in addition. The coefficients aᵢ are calculated so as to obtain the best correlation between the independently determined body composition parameters and the values, determined by the above equation from the impedance values, for the body composition parameter. An overview of various formulas in the form of the above-specified equation is given in the article cited above.

Further factors xᵢ of the above-specified equation for the purpose of improving the determination of the values of the body composition parameter are, for example, the imaginary part of the impedance X_{c}, the weight, age or sex of the subject. An example of an equation, listed in the specified article, for the fat-free body weight (FFM) is:

FFM=−4.104+0.518·Ht²/R50+0.231·weight+0.130·Xc+4.229·sex,

Ht being the overall body size of the subject, and R50 the real part of the impedance over one body side of the subject at 50 kHz, Xc the imaginary part of the impedance, weight the body weight and sex the sex (=1 for men, =2 for women).

The essential factor in many of the formulas used as above is x₁=Ht²/R50. The consideration that lies behind the expression x₁=Ht²/R50 proceeds from the fact that it is possible to use the length of a cylinder and the resistance to draw a conclusion on the volume thereof (R=β·L/A, R being the impedance, ρ the specific resistance, L the length and A the cross-sectional area of the cylinder). Here, however, the overall body size Ht is taken as a measure of the lengths of the extremities (arms and legs), and no account is taken of the fact that the lengths of the various extremities can have different ratios one to another. In particular, owing to the large cross section of the trunk (torso) of the body, the trunk contributes only a very small proportion to the total resistance, but comprises a large part of the body volume, and thereby substantially determines the values to be determined for the body composition parameters.

### SUMMARY

It is an object of the present invention to improve a method and a unit for determining a body composition parameter by bioimpedance measurement as regards its accuracy by taking account of the lengths of the limbs of the subject without having to measure the lengths of the individual limbs. In particular, better account is to be taken of the proportion of the trunk of the subject in the value to be determined for the body composition parameter.

A method for determining a body composition parameter and a unit for implementing the method serve to achieve this object and are described herein and in the claims.

According to one aspect of the present invention, a method is provided for determining a body composition parameter by applying a bioimpedance measurement on a subject, wherein the subject's body includes different body segments and presents an overall body size and a body impedance value. The method comprises the steps of: using the overall body size and the body impedance value in calculating a body composition parameter, and when calculating the body composition parameter, entering a term in the calculation which constitutes the product of a predetermined coefficient and of a ratio of measured impedance values of different body segments of the subject. It has emerged that individually varying length ratios of body segments can be better taken into account in this way.

According to another aspect of the present invention, the body segments include a trunk and a plurality of body extremities, and the method includes the step of: during the calculation of the ratio of impedance values of different body segments, forming the ratio from the impedance value of the trunk and the impedance value of a body extremity.

According to another aspect of the present invention, the body segments include a trunk and a plurality of body extremities, and the method includes the step of: during the calculation of the ratio of impedance values of different body segments, forming the ratio from the impedance value of the trunk and the sum of the impedance values of at least two body extremities.

According to another aspect of the present invention, the body segments include a trunk and a plurality of body extremities, and the method includes the step of: during the calculation of the ratio of impedance values of different body segments, forming the ratio from the impedance value of the trunk and the sum of the impedance values of all body extremities.

According to another aspect of the present invention, during the calculation of the ratio of impedance values of different body segments, the ratio of the real parts of the impedances takes the form of

$${x}_{R Trunk / Extremities} = \frac{{R}_{Trunk}}{( {R}_{Right arm} + {R}_{Left arm} + {R}_{Right leg} + {R}_{Left leg} ) / 4} .$$

According to another aspect of the present invention, during the calculation of the ratio of impedance values of different segments, the ratio of the imaginary parts of the impedances takes the form of

$${x}_{XcTrunk / Extremities} = \frac{{Xc}_{Trunk}}{( {Xc}_{Right arm} + {Xc}_{Left arm} + {Xc}_{Right leg} + {Xc}_{Left leg} ) / 4} .$$

There is no need in the case of the inventive methods to expend effort in measuring the lengths of all body segments individually—rather, instead of this, the impedances of individual body segments are used as a measure. The complex impedance Z for the segments of trunk, right arm, left arm, right leg and left leg is measured at a frequency of preferably 50 kHz. The real part R and imaginary part Xc are then formed from this complex impedance. The following ratios are formed, in turn, from these values:

$${x}_{R 50 trunk / extremities} = \frac{{R}_{50 kHz , trunk}}{( {R}_{50 kHz , right arm} + {R}_{50 kHz , left arm} + {R}_{50 kHz , right leg} + {R}_{50 kHz , left leg} ) / 4}$$

$${x}_{Xc 50 trunk / extremities} = \frac{{Xc}_{50 kHz , trunk}}{( \begin{matrix}{Xc}_{50 kHz , rightarm} + {Xc}_{50 kHz , left arm} + \\ {Xc}_{50 kHz , rightleg} + {Xc}_{50 kHz , left leg}\end{matrix} ) / 4} .$$

These ratios can then enter as x, into the above-stated formula for Value.

However, many variations of these ratios are also conceivable. It is in each case only an exemplary formula that is specified. In particular, they are not described anew for Xc. Moreover, the various approaches can, of course, also be combined.

It would, for example, be possible to consider the values only of one body side:

$${x}_{R 50 trunk / extremities} = \frac{{R}_{50 kHz , trunk}}{( {R}_{50 kHz , right arm} + {R}_{50 kHz , right leg} ) / 2} .$$

It is possible to dispense with the averaging:

$${x}_{R 50 trunk / extremities} = \frac{{R}_{50 kHz , trunk}}{{R}_{50 kHz , right arm} + {R}_{50 kHz , left arm} + {R}_{50 kHz , right leg} + {R}_{50 kHz , left leg}} .$$

Separate indices can be formed for arms and legs:

$${x}_{R 50 trunk / extremities} = \frac{{R}_{50 kHz , trunk}}{( {R}_{50 kHz , right arm} + {R}_{50 kHz , left arm} ) / 2}$$

$${x}_{R 50 trunk / extremities} = \frac{{R}_{50 kHz , trunk}}{( {R}_{50 kHz , rightleg} + {R}_{50 kHz , left leg} ) / 2} .$$

It is possible to take the ratio of arm or leg relating to the body half:

$${x}_{R 50 arm / side} = \frac{{R}_{50 kHz , right arm} + {R}_{50 kHz , left arm}}{\begin{matrix}{R}_{50 kHz , right arm} + {R}_{50 kHz , rightleg} + {R}_{50 kHz , trunk} + \\ {R}_{50 kHz , left arm} + {R}_{50 kHz , left leg} + {R}_{50 kHz , trunk}\end{matrix}}$$

$${x}_{R 50 leg / side} = \frac{{R}_{50 kHz , right leg} + {R}_{50 kHz , left leg}}{\begin{matrix}{R}_{50 kHz , right arm} + {R}_{50 kHz , rightleg} + {R}_{50 kHz , trunk} + \\ {R}_{50 kHz , left arm} + {R}_{50 kHz , left leg} + {R}_{50 kHz , trunk}\end{matrix}} .$$

It would be possible to take trunk+leg as measure for the body length and to form the ratio of arms and legs to this end:

$${x}_{R 50 arm / ( leg + trunk )} = \frac{{R}_{50 kHz , right arm} + {R}_{50 kHz , left arm}}{{R}_{50 kHz , rightleg} + {R}_{50 kHz , trunk} + {R}_{50 kHz , left leg} + {R}_{50 kHz , trunk}}$$

$${x}_{R 50 leg / ( leg + trunk )} = \frac{{R}_{50 kHz , rightleg} + {R}_{50 kHz , left leg}}{{R}_{50 kHz , rightleg} + {R}_{50 kHz , trunk} + {R}_{50 kHz , left leg} + {R}_{50 kHz , trunk}} .$$

It is possible to calculate using the reciprocal:

$${x}_{R 50 extremities / trunk} = \frac{( \begin{matrix}{R}_{50 kHz , rightarm} + {R}_{50 kHz , left arm} + \\ {R}_{50 kHz , rightleg} + {R}_{50 kHz , left leg}\end{matrix} ) / 4}{{R}_{50 kHz , trunk}} .$$

Instead of the real part and imaginary part it would be possible to use the absolute value:

$${x}_{Z 50 trunk / extremities} = \frac{\vert {Z}_{50 kHz , trunk} \vert}{( \frac{\vert {Z}_{50 kHz , rightarm} \vert + \vert {Z}_{50 kHz , left arm} \vert +}{\vert {Z}_{50 kHz , rightleg} \vert + \vert {Z}_{50 kHz , left leg} \vert} ) / 4} .$$

According to another aspect of the present invention, a bioimpedance measurement unit is provided. The unit includes a plurality of electrodes for making contact with a subject having a body including different body segments and presenting an overall body size and a body impedance value. The unit also includes a control and evaluation unit. The control and evaluation unit is configured to inject alternating current via a plurality of said electrodes and to measure resulting voltages via others of said electrodes, in order to determine the impedances of the body and individual body segments therefrom. The control and evaluation unit is further configured to determine a body composition parameter, wherein a term is entered which constitutes the product of a predetermined coefficient and of a ratio of measured impedance values of different body segments of the subject, and the overall body size and the body impedance value are used in the calculation of the body composition parameter.

This summary is provided to introduce a selection of concepts in a simplified form. These concepts are further described below in the detailed description of the preferred embodiments. This summary is not intended to identify key features or essential features of the claimed subject matter, nor is it intended to be used to limit the scope of the claimed subject matter.

Various other aspects and advantages of the present invention will be apparent from the following detailed description of the preferred embodiments and the accompanying drawing figures.

### BRIEF DESCRIPTION OF THE DRAWING FIGURES

Preferred embodiments of the present invention are described in detail below in conjunction with the attached drawing figures, in which:

FIG. 1 is a schematic of a subject as a connection diagram for a bioimpedance measurement method;

FIG. 2 is a schematic of a bioimpedance measurement unit;

FIG. 3 shows a distribution of the deviations of the body composition parameter of lean soft tissue mass (LST) of the right leg, and the deviations of the LST values determined by the bioimpedance measurement from independently determined LST values for a representative subject group, a method according to the prior art having been applied to determine the body composition value by bioimpedance measurement; and

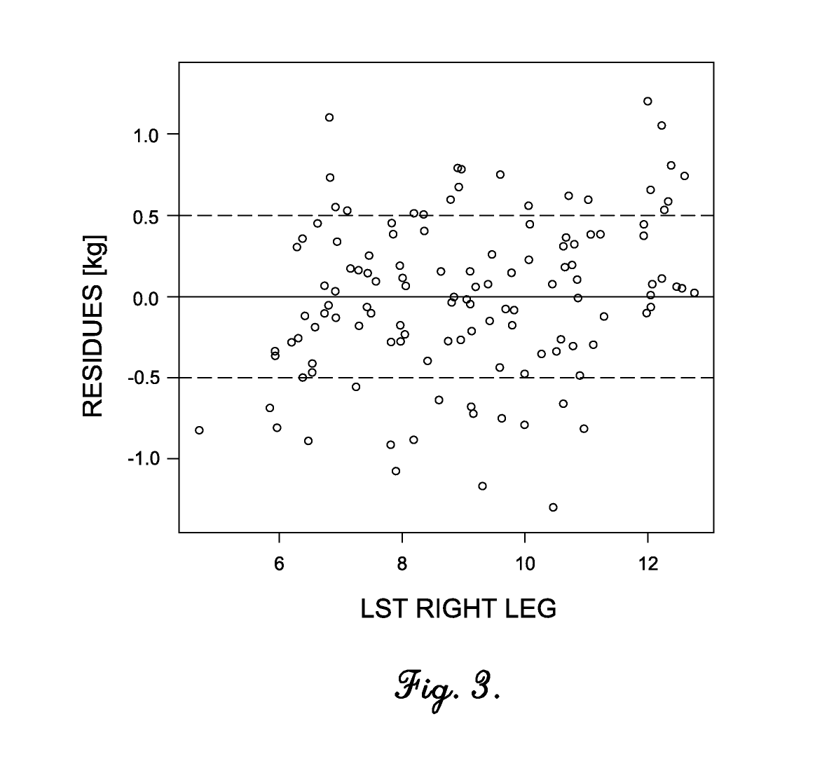

FIG. 4 shows the same distribution of the deviations of the body composition values LST as shown in FIG. 3, the values of the body composition parameter having been determined here by bioimpedance measurement using a method in accordance with the present invention.

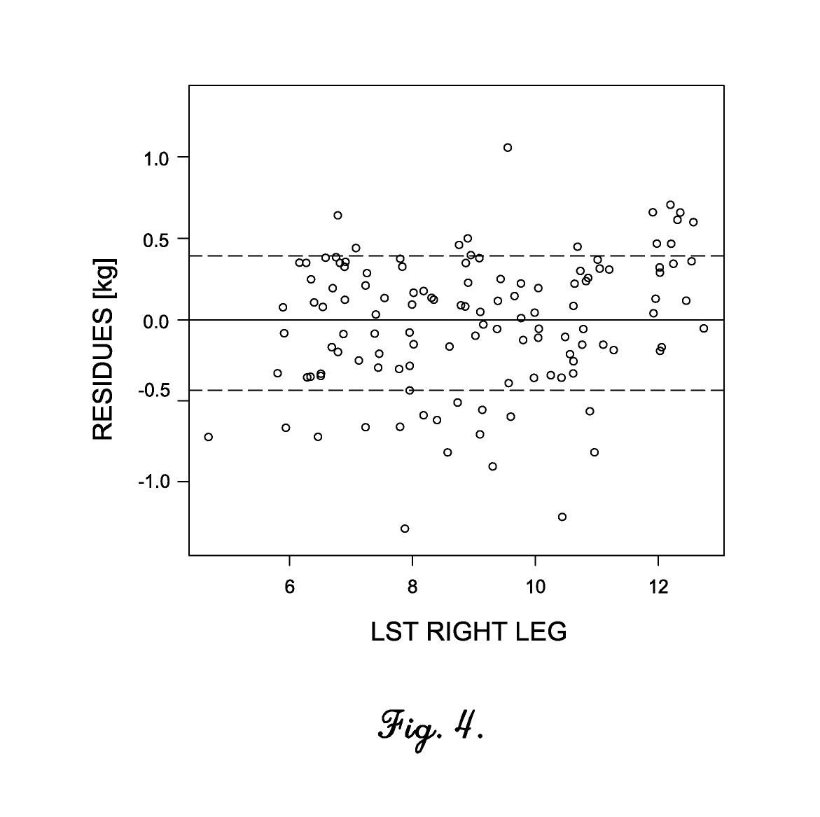

The drawing figures do not limit the present invention to the specific embodiments disclosed and described herein. The drawings are not necessarily to scale, emphasis instead being placed upon clearly illustrating the principles of the preferred embodiments.

### DETAILED DESCRIPTION OF THE PREFERRED EMBODIMENTS

The present invention is susceptible of embodiment in many different forms. While the drawings illustrate, and the specification describes, certain preferred embodiments of the invention, it is to be understood that such disclosure is by way of example only. There is no intent to limit the principles of the present invention to the particular disclosed embodiments. Furthermore, although an exhaustive description is not provided herein of the numerous embodiments which might be made through the combination of features selected from the embodiments described below, such hybrid or combined embodiments fall entirely within the scope of the present invention.

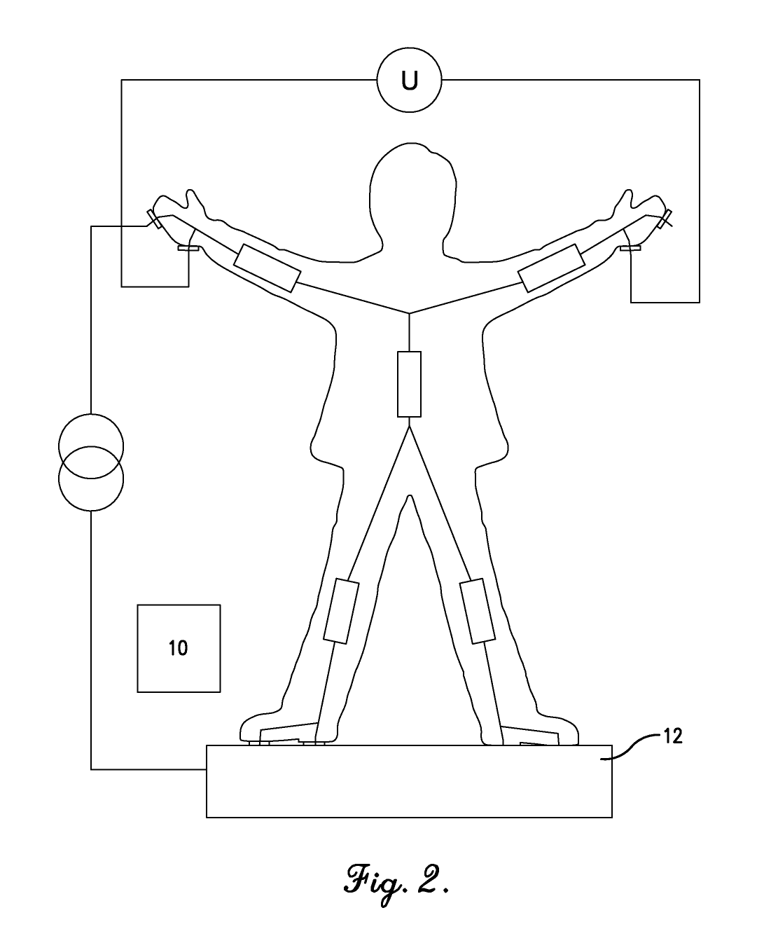

A typical unit for bioimpedance measurements is shown in FIG. 2, and has, for example, a standing platform 12 on which the person to be measured places himself with both feet. Two electrodes are designed for each foot on the standing platform 12, for example one in the region of the heel and one in the region of the forefoot. Also present are two handles which the user grips in a specific way and thereby bring each hand into contact with two electrodes, for example respectively one on the index and middle fingers and one on the small finger and ring finger.

A control and evaluation unit 10 is set up to carry out various measurement programs which can be used to carry out impedance measurements for the entire body, a body side or individual body segments. When, for example, an alternating current is injected via electrodes on a hand and a foot of the same body side, and the resulting voltage on the same hand and the same foot is measured with the respective other electrode, the impedance of an entire body side is measured, that is to say a measure for the total body impedance is obtained.

In another measurement program, alternating current is injected, in turn, via an electrode on a hand and an electrode on a foot of the same body side, and the voltage at the electrode of one hand and the other hand is measured; this situation is shown in FIG. 2. This impedance measurement is sensitive to the impedance of the arm through which current is flowing. In a further measurement program, alternating current is injected in the same way as previously, and the voltage is measured via an electrode on one foot and an electrode on the other foot. This impedance measurement is sensitive to the impedance of the leg through which current is flowing.

The impedance values of the entire body and individual body segments can be measured in this way.

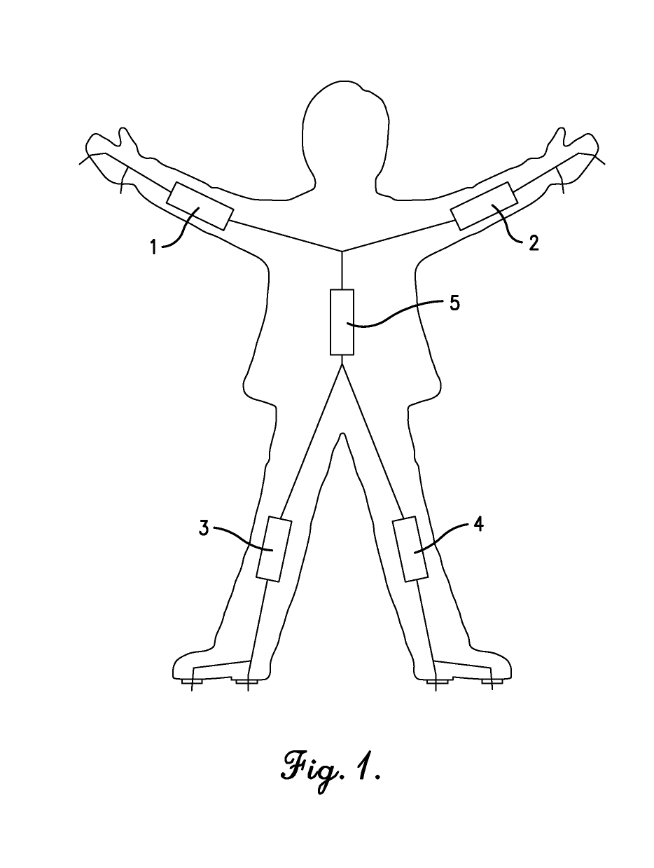

The impedances of the body segments are denoted as follows in the equivalent connection diagram in FIG. 1:
1: impedance left arm
2: impedance right arm
3: impedance left leg
4: impedance right leg
5: impedance trunk.

The impedances are complex values that are determined at a specific frequency, for example at 50 kHz. The symbol Z is used for the complex impedance, R denotes the real part of Z and Xc denotes the imaginary part of Z. Specified in kHz in the index is the frequency at which the impedance was determined, and the body segment on which it was measured.

Consideration is given below to a measurement on a group of subjects on which there were respectively undertaken an impedance measurement and an independent measurement of the body composition parameter of lean soft tissue (LST) mass of the right leg. A DXA (dual energy x-ray absorption) measurement was carried out for the independent measurement of this body composition parameter. Subsequently, in order to predict the body composition value LST use was made of an equation of the form:

LST_{rightleg}=a₁·x₁+a₂·x₂+a₃+x₃+a₄·x₄,

wherein x₁=Ht²/R50_{right leg}, x₂=Xc50_{right leg}, x₃=weight and x₄=sex. A multilinear regression was then undertaken in order to determine the coefficients aᵢ that yield the best possible correlation of the body composition parameter LST_{right leg} from the bioimpedance measurement and the independent measurement. A coefficient of determination r²=0.937 is obtained with this method according to the prior art. The coefficient of determination r² is the square of the correlation coefficient r, and specifies which component of the variance of LST_{right leg} according to the above formula is explained by the linear regression. The coefficient of determination lies between 0 (no linear relationship) and 1 (perfect linear relationship). The distribution of the differences between the value of LST_{right leg} according to the above formula and that determined by independent measurement has a standard deviation of 0.501 kg, which can also be regarded as an uncertainty or error.

If the following ratio is added as x₅, namely

$${x}_{5} = \frac{{R}_{trunk}}{( {R}_{right arm} + {R}_{left arm} + {R}_{right leg} + {R}_{left leg} ) / 4}$$

and an associated coefficient a₅ is determined by regression, the coefficient of determination r² is improved with the aid of an inventive method to 0.948, and the error (standard deviation of the distribution of the differences between calculated LST_{right leg} and independently measured) is improved to 0.459 kg.

If use is made, in addition, of

$${x}_{6} = \frac{{Xc}_{trunk}}{( {Xc}_{right arm} + {Xc}_{left arm} + {Xc}_{right leg} + {Xc}_{left leg} ) / 4}$$

as x₆ and of an associated coefficient a₆ determined by regression, the result is a further improvement to the coefficient of determination r² to 0.956, and of the error to 0.425 kg. This demonstrates that the inventively provided procedure yields an improvement in the measuring accuracy for body composition parameters with the aid of bioimpedance measurement.

Although the above description presents features of preferred embodiments of the present invention, other preferred embodiments may also be created in keeping with the principles of the invention. Furthermore, as noted previously, these other preferred embodiments may in some instances be realized through a combination of features compatible for use together despite having been presented independently as part of separate embodiments in the above description.

The preferred forms of the invention described above are to be used as illustration only and should not be utilized in a limiting sense in interpreting the scope of the present invention. Obvious modifications to the exemplary embodiments, as hereinabove set forth, could be readily made by those skilled in the art without departing from the spirit of the present invention.

The inventors hereby state their intent to rely on the Doctrine of Equivalents to determine and assess the reasonably fair scope of the present invention as pertains to any apparatus not materially departing from but outside the literal scope of the invention set forth in the following claims.

What is claimed is:

1. A method for determining a body composition parameter by applying a bioimpedance measurement on a subject, wherein the subject's body includes different body segments and presents an overall body size and a body impedance value, said method comprising the steps of:
using the overall body size and the body impedance value in calculating a body composition parameter, and
when calculating the body composition parameter, entering a term in the calculation which constitutes the product of a predetermined coefficient and of a ratio of measured impedance values of different body segments of the subject.

2. The method as claimed in claim 1,
said body segments including a trunk and a body extremity,
during the calculation of the ratio of impedance values of different body segments, forming the ratio from the impedance value of the trunk and the impedance value of a body extremity.

3. The method as claimed in claim 1,
said body segments including a trunk and a plurality of body extremities,
during the calculation of the ratio of impedance values of different body segments, forming the ratio from the impedance value of the trunk and the sum of the impedance values of at least two body extremities.

4. The method as claimed in claim 3,
during the calculation of the ratio of impedance values of different body segments, forming the ratio from the impedance value of the trunk and the sum of the impedance values of all body extremities.

5. The method as claimed in claim 4,
during the calculation of the ratio of impedance values of different body segments, the ratio of the real parts of the impedances takes the form of
$${x}_{R Trunk / Extremities} = \frac{{R}_{Trunk}}{( {R}_{Right arm} + {R}_{Left arm} + {R}_{Right leg} + {R}_{Left leg} ) / 4} .$$

6. The method as claimed in claim 4,
during the calculation of the ratio of impedance values of different body segments, the ratio of the imaginary parts of the impedances takes the form of
$${x}_{XcTrunk / Extremities} = \frac{{Xc}_{Trunk}}{( {Xc}_{Right arm} + {Xc}_{Left arm} + {Xc}_{Right leg} + {Xc}_{Left leg} ) / 4} .$$

7. The method as claimed in claim 1,
wherein the impedance values are measured at a frequency of 50 kHz.

8. A bioimpedance measurement unit comprising:
a plurality of electrodes for making contact with a subject having a body including different body segments and presenting an overall body size and a body impedance value; and
a control and evaluation unit,
said control and evaluation unit being configured to inject alternating current via a plurality of said electrodes and to measure resulting voltages via others of said electrodes, in order to determine the impedances of the body and individual body segments therefrom,
wherein the control and evaluation unit is further configured to determine a body composition parameter, wherein a term is entered which constitutes the product of a predetermined coefficient and of a ratio of measured impedance values of different body segments of the subject, and the overall body size and the body impedance value are used in the calculation of the body composition parameter.
